(19) United States
(12) Patent Application Publication
(10) Pub. No.: US 2010/0249900 A1
(43) Pub. Date: Sep. 30, 2010

(54) Drug eluting medical implant with porous surface

(75) Inventors: Laura Sager, Zuerich (CH); Bjoern Klocke, Zuerich (CH); Heinz Mueller, Erlangen (DE); Nina Adden, Nuernberg (DE); Gerhard Kappelt, Erlangen (DE)

Correspondence Address: BIOTECH BEACH LAW GROUP , PC, 5677 OBERLIN DRIVE, SUITE 204, SAN DIEGO, CA, 92121, US

(73) Assignee: BIOTRONIK VI Patent AG, Baar (CH)

(21) Appl. No.: 12/730,472

(22) Filed: Mar. 24, 2010

(30) Foreign Application Priority Data
Mar. 26, 2009 (DE) 10 2009 001 895.6

(51) Int. Cl. A61F 2/82

(52) U.S. Cl. 623/1.15; 623/1.46; 623/1.39

(57) ABSTRACT

An implant is proposed, in particular made from a base material including a biodegradable metal and/or a biodegradable metal alloy, wherein the implant includes a coating made of crystalline calcium phosphate and/or amorphous calcium phosphate.

### CROSS REFERENCE TO RELATED APPLICATIONS

This invention claims benefit of priority to Germany patent application serial number DE 10 2009 001 895.6, filed on Mar. 26, 2009; the contents of which is herein incorporated by reference in its entirety.

### FIELD OF THE INVENTION

The invention relates to a medical implant, preferably an absorbable metal stent (AMS) with a coated surface.

### BACKGROUND OF THE INVENTION

Arteriosclerosis is a wide spread disease of human arteries. Various types of substances called plaque accumulate at the arterial walls and restrict the native blood flow of the vessel. If the disease progresses, patients risk ischemia driven malfunctions of parts of the body that get their blood supply via the respective vessel.

There are various forms of treatment of plaque lesions like bypass surgery and artherectomy. One of the most wide spread treatments are non-invasive procedures involving balloon catheters for opening the lesion and stent for scaffolding the re-opened artery and preventing vessel recoil.

Stents are traditionally made of permanent metals such as 316L and Nitinol. Over the last years, degradable stents were developed that shall bioabsorb after the scaffolding function of the stent is not longer needed. In particular, Magnesium alloy stents were developed and clinically studied in the coronaries. It has been shown that those sterns are safe but degrade before the recoil tendency of the vessel stops. Hence those stents suffered from vessel recoil (as previously known from pure balloon angioplasty without stenting). Also it could be shown that there is tissue formation inside the stent (called neointima proliferation) that is also known from permanent stents.

Clinically successful magnesium alloy stents need to exhibit longer scaffolding ability than known today, and also benefit from reduced neointima proliferation.

Surface modifications have been proposed to reduce magnesium alloy stent degradation and thereby prolong scaffolding time. These include anorganic coatings include phosphates, oxides and others. Experiments have shown that these coatings are effective to io decrease degradation rates of undeformed specimen. However, stents, which are crimped on a catheter prior to implantation and are expanded at the treatment site inside the human body, face strong surface deformation (usually 20-40%) of the metallic stent body, and the anorganic coating get cracks and embrittlement, which render the degradation control effect ineffective.

One of the anorganic coatings known is hydroxy apatite. Today hydroxy apatite is well known as an implant material that shows superior biocompatibility in contact with most kinds of human tissue. Therefore hydroxy apatite has found a large range of applications in implantology mostly as a coating and mostly for orthopaedic or traumatologic implants. In these applications implant surfaces can he coated with hydroxy apatite by many deposition processes like plasma spraying processes, chemical vapour deposition processes, sintering processes, electrochemical processes or many more. All of these processes deposit these hydroxy apatite coatings on the original implant surface or on a primer layer thereon. Unfortunately, hydroxy apatite coatings turned out to he ineffective in stent degradation control for its lack to follow the high elastic deformation as needed for typical stents.

Neointima control for permanent stents is typically achieved by drug-elution coatings on the stents. Typically anti-proliferative drugs such as Paclitaxel or Sirolimus and their derivatives are applied embedded, i.e., mixed with drug carriers of permanent polymers (such as Polysulphone and SIBS) or degradable polymers (polyesters such as PLLA, PCL and PLGA). However, these techniques can not he applied to magnesium alloy stents. Permanent polymers are blown up by the hydrogen evolution from magnesium surfaces, and degradable polyesters speed up the magnesium degradation process unfavorably by their acidic break-down. More specialized, non-polyester type degradable drug-elution coatings have been proposed but these coatings typically lack adhesion to magnesium surfaces.

The draw-backs of a drug eluting absorbable metal stent (DREAMS) with a normal permanent drug carrier are: no full degradation of stent, and problems with hydrogen evolution (although hydrogen evolution is not related to the coating but to the base material).

The draw-backs of DREAMS with a normal degradable polymeric drug carrier (PLLA, PLGA etc. and polyesters) are: bad biological interaction, swelling of polymer (therefore no long protection of Mg against body fluids), no proven safety against subacute thromboses (even with degradable polymers), and issues with acidic break-down of the polyesters.

The draw-backs of DREAMS with a lipid drug carrier are: had adhesion of (soft/rather liquid) carrier, and often release is too quick.

The draw-back of DREAMS with a porous surface and drug in it is that the elution kinetic is too fast.

### DESCRIPTION OF THE INVENTION

The object of the present invention is to overcome these drawbacks. This object is achieved by a medical implant, such as a stent, made from a base material including a biodegradable metal and/or a biodegradable metal alloy, wherein the implant includes a coating made of crystalline calcium phosphate and/or amorphous calcium phosphate.

The calcium phosphate coating may be achieved by a coating with hydroxyapatite, a biological apatite, dicalcium phosphates, tricalcium phosphates, fluoroapatite, chloroapatite or a compound consisting mostly of calcium phosphate in a hydroxy apatite like or hydroxy apatite near stochiometric composition.

The mentioned coating of the implant may contain traces or precipitations of the base material of the implant. The base material is magnesium or a magnesium alloy, preferably the magnesium alloy is WE43.

The magnesium alloy may contain one alloying element up to 50 At % out of the group including calcium, zinc, manganese. lithium, iron or up to 50 At % of a combination thereof.

The magnesium alloy may contain additionally up to 20 At % yttrium or other rare earth elements. In this case the precipitations include yttrium and/or other rare earth metals, such as neodymium. The precipitations have, for example, a size of 0.5-3 microns for yttrium and 1-7 microns for neodymium.

In a preferred embodiment the calcium phosphate coating has a layered structure. The first layer that directly contacts the medical implant is a crystallized calcium phosphate layer. This is a substantially dense layer which controls and delays the degradation of the underlying degradable material of the implant. Above this crystalline, mostly dense layer is applied an amorphous calcium phosphate layer.

Preferably at least one of the layers of the coating has a layer thickness of 0.1 to 5 microns.

In a preferred embodiment the amorphous calcium phosphate layer has pores with a pore size between 0.02 and 50 microns. In case that the porous layer consists of hydroxy apatite the pore size is preferably in the range of 0.02 to 1 micron.

The porous layer of the implant may be filled with one or more lipids, preferably fatty acid, mono-, di-, or triacylglycerides, waxes, phospholipids, sphingolipids, steroids and degradable polymers showing non-acidic break-down mechanism.

Additionally a drug can be embedded either in the amorphous calcium phosphate layer or in the lipid component. The drug or the drugs can be all anti-inflammatory and/or anti-proliferative drugs, including, Paclitaxel, Sirolimus and its true analogues and/or antimycotic, etc. Also the usage of multiple drugs is possible.

The antiproliferative, anti-inflammatory and/or antimycotic drug may be chosen from the following list: Abciximab, Acemetacin, Acetylvismion B, Aclarubicin, Ademetionin, Adriamycin, Aescin, Afromoson, Akagerin, Aldesleukin, Amidoron, Aminoglutethemid, Amsacrin, Anakinra, Anastrozol, Anemonin, Anopterin, Antimykotika, Antithrombotika, Apocymarin, Argatroban, Aristolactam-All, Aristolochsäure, Ascomyein, Asparaginase, Aspirin, Atorvastatin, Auranofin, Azathioprin, Azithromycin, Baccatin, Bafilomycin, Basiliximab, Bendamustin, Benzocain, Berberin, Betulin, Betulinsäure, Bilobol, Bisparthenolidin, Bleomycin, Bombrestatin, Boswellinsäuren and their derivates, Bruceanole A,B and C, Bryophyllin A, Busulfan, Antithrombin, Bivalirudin, Cadherine, Camptothecin, Capecitabin, o-Carbamoylphenoxyessigsäure, Carboplatin, Carmustin, Celecoxib, Cepharantin, Cerivastatin, CETP-Inhibitoren, Chlorambucil, Chloroquinphosphat, Cictoxin, Ciprolloxacin, Cisplatin, Cladribin, Clarithromycin, Colchicin, Concanamycin, Coumadin, C-Type Natriuretic Peptide (CNP), Cudraisoflavon A, Curcumin, Cyclophosphamid, Cyclosporin A, Cytarabin, Dacarbazin, Daclizumab, Dactinomycin, Dapson, Daunorubicin, Diclofenac, 1,11-Dimethoxycanthin-6-on, Docetaxel, Doxorubicin, Dunaimycin, Epirubicin, Epothilone A and B, Erythromycin, Estramustin, Etobosid, Everolimus, Filgrastim, Fluroblastin, Fluvastatin, Fludarabin, Fludarabin-5′-dihydrogenphosphat, Fluorouracil, Folimycin, Fosfestrol, Gemcitabin, Ghalakinosid, Ginkgol, Ginkgolsäure, Glykosid 1a, 4-Hydroxyoxycyclophosphamid, Idarubicin, Ifosfamid, Josamycin, Lapachol, Lomustin, Lovastatin, Melphalan, Midecamycin, Mitoxantron, Nimustin, Pitavastatin, Pravastatin, Procarbazin, Mitomycin, Methotrexat, Mereaptopurin, Thioguanin, Oxaliplatin, Irinotecan, Topotecan, Hydroxycarbamid, Miltefosin, Pentostatin, Pegasparase, Exemestan, Letrozol, Formestan, SMC-Proliferation-Inhibitor-2w, Mitoxanthrone, Mycophenolatmofetil, c-myc-Antisense, [beta]-myc-Antisense, [beta]-Lapachon, Podophyllotoxin, Podophyllsäure-2-ethylhydrazid, Molgramostim (rhuGM-CSF), Peginterferon [alpha]-2b, Lanograstim (r-HuG-CSF), Macrogol, Selectin (Cytokinantagonist), Cytokininhibitoren, COX-2-Inhibitor, NFkB, Angiopeptin, monoclonale Antikörper, which block the mussel cell proliferation, bFGF-Antagonists, Probucol, Prostaglandine, 1-Hydroxy-11-Methoxycanthin-6-on, Scopolectin, NO-Donors as Pentaerythrityltetranitrat and Syndnoeimine, S-Nitrosoderivate, Tamoxifen, Staurosporin, [beta]-Estradiol, [alpha]-Estradiol, Estriol, Estron, Ethinylestradiol, Medroxyprogesteron, Estradiolcypionate, Estradiolbenzoate, Tranilast, Kamebakaurin and other Terpenoide used in cancer therapy, Verapamil, Tyrosin-Kinase-Inhibitoren (Tyrphostine), Paclitaxel and their derivates as 6-[alpha]-Hydroxy-Paclitaxel, Taxotere, Kohlensuboxids (MCS) and their macrocyclic Oligomers, Mofebutazon, Lonazolac, Lidocain, Ketoprofen, Mefenaminsäure, Piroxicam, Meloxicam, Penicillamin, Hydroxychloroquin, Natriumaurothiomalat, Oxaceprol, [beta]-Sitosterin, Myrtecain, Polidocanol, Nonivamid, Levomenthol, Ellipticin, D-24851 (Calbiochem), Colcemid, Cytochalasin A-E, Indanocine, Nocadazole, S 100 Protein, Bacitracin, Vitronectin-Rezeptor Antagonisten, Azelastin, Guanidylcyclase-Stimulator Gewebsinhibitor der Metallproteinase-1 und 2, free Nukleinsäuren, Nukleinsäuren in Virenüberträger inkorporiert, DNA- und RNA-Fragmente, Plaminogen-Aktivator Inhibitor-1, Plasminogen-Aktivator Inhibitor-2, Antisense Oligonucleotide, VEGF-Inhibitoren, IGF-1, drugs from the group of Antibiotica as Cefadroxil, Cefazolin, Cefaclor, Cefotixin Tobramycin, Gentamycin, Penicilline as Dicloxacillin, Oxacillin, Sulfonamide, Metronidazol, Enoxoparin, desulfatiertes and N-reacetyliertes Heparin (Hemoparin<(R)>), Gewebe-Plasminogen-Aktivator, GpIlb/Illa-Plättchenmembranrezeptor, Faktor Xa-Inhibitor Antikörper, Heparin, Hirudin, r-Hirudin, PPACK, Protamin, Prourokinase, Streptokinase, Warfarin, Urokinase, Vasodilatoren as Dipyramidol, Trapidil, Nitroprusside, PDGF-Antagonisten as Triazolopyrimidin and Seramin, ACE-Inhibitoren wie Captopril, Cilazapril, Lisinopril, Enalapril, Losartan, Thioproteaseinhibitoren, Prostacyclin, Vapiprost, Interferon [alpha], [beta] und [gamma], Histaminantagonisten, Serotoninblocker, Apoptoseinhibitoren, Apoptoseregulatoren as p65, NF-kB oder Bcl-xL-Antisense-Oligonukleotiden, Halofuginon, Nifedipin, Tocopherol Tranilast, Molsidomin, Teepolyphenole, Epicatechingallat, Epigallocatechingallat, Leflunomid, Etanercept, Sulfasalazin, Etoposid, Dicloxacyllin, Tetracyclin, Triamcinolon, Mutamycin, Procainimid, Retinolsäure, Quinidin, Disopyrimid, Flecainid, Propafenon, Sotolol, natural and synthetic Steroids as Inotodiol, Maquirosid A, Ghalakinosid, Mansonin, Streblosid, Hydrocortison, Betamethason, Dexamethason, nonsteroid substances (NSAIDS) such as Fenoporfen, Ibuprofen, Indomethacin, Naproxen, Phenylbutazon an other antivirale Agentien as Acyclovir, Ganciclovir and Zidovudin, Clotrimazol, Flucytosin, Griseofulvin, Ketoconazol, Miconazol, Nystatin, Terbinafin, antiprozoale Agentien as Chloroquin, Mefloquin, Quinin, natural Terpenoide as Hippocaesculin, Barringtogenol-C21-angelat, 14-Dehydroagrostistachin, Agroskerin, Agrostistachin, 17-Hydroxyagrostistachin, Ovatodiolide, 4,7-Oxycycloanisomelsäure, Baccharinoide B1, B2, B3 and B7, Tubeimosid, Bruceantinoside C, Yadanzioside N, and P, Isodeoxyelephantopin, Tomenphantopin A and B, Coronarin A,B,C and D, Ursolsäure, Hyptatsäure A, Iso-Iridogermanal, Maytenfoliol, Effusantin A, Excisanin A and B, Longikaurin B, Sculponeatin C, Kamebaunin, Leukamenin A and B, 13,18-Dehydro-6-alpha-Senecioyloxychaparrin, Taxamairin A and B, Regenilol, Triptolid, Cymarin, Hydroxyanopterin, Protoanemonin, Cheliburinchlorid, Sinococulin A and B, Dihydronitidin, Nitidinchlorid, 12-beta-Hydroxypregnadien 3,20-dion, Helenalin, Indicin, Indicin-N-oxid, Lasiocarpin, Inotodiol, Podophyllotoxin, Justicidin A and B, Larreatin, Malloterin, Mallotochromanol, Isobutyrylmallotochromanol, Maquirosid A, Marchantin A, Maytansin, Lycoridicin, Margetin, Pancratistatin, Liriodenin, Bispsrthenolidin, Oxoushinsunin, Periplocosid A, Ursolsäure, Deoxypsorospermin, Psycorubin, Ricin A, Sanguinarin, Manwuweizsäure, Methylsorbifolin, Sphatheliachromen, Stizophyllin, Mansonin, Streblosid, Dihydrousambaraensin, Hydroxyusambarin, Strychnopentamin, Strychnophyllin, Usambarin, Usambarensin, Liriodenin, Oxoushinsunin, Daphnoretin, Lariciresinol, Methoxylarieiresinol, Syringaresinol, Sirolimus (Rapamycin), Somatostatin, Tacrolimus, Roxithromycin, Troleandomycin, Simvastatin, Rosuvastatin, Vinblastin, Vincristin, Vindesin, Teniposid, Vinorelbin, Tropfosfamid, Treosulfan, Tremozolomid, Thiotepa, Tretinoin, Spiramycin, Umbelliferon, Desacetylvismion A, Vismion A and B, Zeorin.

Also, the lipid might be used to embed pH-buffer substances to control degradation and/or to protect the drug further from high pH values created by the Mg degradation.

Biodegradable metallic implants possess favourable mechanical and biological properties so but tend to erode too fast, resulting in a complete or nearly complete degradation before the end of the treatment time. The aim of the present invention is to provide a coating to control such degradation rate. A further aspect of the present invention is directed to a therapeutic agent that may be incorporated into the coating of that medical device.

In the past, polymers have been used as coating to control the degradation of biodegradable stents. Unfortunately, when polymers are used. the degradation process is often accompanied by inflammatory response of the tissue. Hydroxy apatite coating, as a type of calcium phosphate. is characterized by being entirely biocompatible.

Even biodegradable metallic implants materials generate layers of reaction/conversion io products that arc similar to the compositions of apatites when they degrade in physiological solutions. Apatite layers can result by a pre-reaction of the implant surface in a physiological solution or related solutions. This gives an especially high bonding strength of the coating to the substrate. One characteristic of hydroxy apatite layers produced in that way is that they contain certain amounts or traces of the former base material that has been transformed. This could for example be traces of Magnesium in the case of processing absorbable Magnesium implants or alloying elements or precipitations from the base metal like for example rare earth metals or precipitations containing rare earth metals when processing implants made from rare earth containing Magnesium alloys.

Surprisingly it was discovered that calcium phosphate layers have a lot of interesting features beside good biocompatibility. So it has been found, that these coatings have corrosion inhibiting effects. It also has been found, that many of these coatings have a rough or fissured or porous structure. The individual appearance of the coating mostly depends on the process used and the parameters of this process. One important parameter is the crystallinity of the calcium phosphate phase. More crystalline phases are more slowly resorbed by the body. Hence, beside coating thickness, crystallinity is a way to control the passivating effect of the calcium phosphate coating.

Regarding implants that have to undergo plastic deformation, brittle apatite layers might crack and therefore lose their protecting properties by enabling fast local corrosion of the substrate. Beside having films with a film thickness lower than 1 μm, to avoid such effects, certain organic substances like lipids (fats, oils, etc.) or certain biodegradable polymers with non-acidic break-down can be used to build up an elastic coating on the calcium phosphate coating that protects the base material from accelerated corrosion or degradation even further and maintains certain degradation intervals for the degradable implant. Lipids are fatty acid, mono- di- or triacylglycerides, waxes, phospholipids, sphingolipids, and steroids just to name a few. Lipids have the advantage that they are highly hydrophobic, therefore, do not swell in water, and protect the AMS longer against the body fluid and therefore postpone degradation of the stent. In other words, such lipids provide an elastic sealing to prevent cracks in the hydroxy apatite to have an unfavorable, uncontrolled effect on stent degradation control.

In addition, such coatings of fats or oils or (biodegradable) polymers can be used very effectively as drug bearing substances.

Some special advantages of the inventive solution. as described above, include: the sealing is of the bottom of the pores together with the use of AMS as a stent platform, the use of the layer not only for its pores and drug attachment but also the degradation control properties for the underlying Mg stent structure, and the coating contains traces from metallic substrate.

In one embodiment, an AMS based on WE 43 has porous hydroxy apatite coating (ca. 0.1-5 μm porous plus separation of 0.1-5 μm with limited water diffusion) filled with hydrogenated soy oil and Paclitaxel (at 20% of TAXUS load, or about 0.2 μg/mm2), hydroxy apatite giving pores but also separating the Mg surface from the carrier (to minimize had interactions between OH— ions and lipids and drug), the pore size (about 0.02-1 μm or rather separated islands depending on the desired release kinetic).

In another embodiment, there is an additional top coat to seal the surface of the stent and potentially additional control of drug release, e.g., top coatings consisting of degradable polymers such as polyesters (e.g. PLGA and PLLA).

In another embodiment, there is a separation layer instead of, or in addition to filled bottom of pores for separation of Mg and lipid/drug (e.g., Silicon Carbide, diamond like carbon).

All other degradable metal stents include various Mg alloys, in general, permanent stents and polymer stents; alloy examples are AZ 31, AZ61, AZ63, AZ80, AZ91, AZ92, WE54, EA55and EA65.

In another embodiment, the lipid has an additional pH buffer embedded (e.g., Calcium carbonate, Sodiumhydrogenphosphate, Calciumphosphates, Sodiumbicarbonate, etc.).

In another embodiment, other lipids and liquid carriers other than soy oil, e.g., cholesterol, saturated fats. oil blends, fatty acids, ester of fatty acids, phospholipids, mono-, di-, triacylglycerides, etc., are used.

Non-limiting examples of crystalline phases of calcium phosphate are: Mono-CaP-Monohydrate (MCPM) Ca(H₂PO₄)₂.H₂O, Di-CaP-Dihydrate (DCPD) CaHPO₄.2H₂O, Octa-CaP (OCP) Ca₈H₂(PO₄)₆.5H₂O, Tri-CaP(TCP)Ca₃(PO₄)₂(.2H₂O) Hydroxyapatite (OHAp) Ca₁₀(PO₄)₆ (OH)₂, Tetra-CaP (TTCP) Ca₄(PO₄)₂O; Fluorapatite; and Chloroapatite. Crystalline growth can occur in many different shapes, e.g. needles, globes, or flat layers.

An example for the manufacturing stent process is the following: (1) Clean AMS surface; (2) Coat hydroxy apatite in electrolyte solution on the stent surface (plasma processing, dip coating, plasma spraying processes, chemical vapour deposition processes, sintering processes. electrochemical processes, pre-corrosion of AMS surface e.g. in artificial plasma as other possibility); (3) Dry: (4) Dip coating of stent in solution consisting of carrier/drug plus solvent; and (4) Dry solvent.

The following is a background for alternative manufacturing of the calcium phosphate (CaPO4) surface: pre-reaction of the implant surface in a physiological solution or related solutions (layers produced in that way contain certain amounts or traces of the former base material that has been transformed). Alternative manufacturing may also include the combination of creating a first layer by an in situ reaction (pre-reaction, reaction of the original material of the stent body) and additional coating by one of the above mentioned processes.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is

1. A medical implant made from a base material comprising a biodegradable metal and/or a biodegradable metal alloy, wherein the implant comprises a coating made of crystalline calcium phosphate and/or amorphous calcium phosphate.

2. The implant according to claim 1, wherein the coating contains at least traces or precipitations of the base material of the implant.

3. The implant according to claim 1, wherein the base material of the implant is magnesium or a magnesium alloy.

4. The implant according to claim 3, wherein the magnesium alloy contains up to 50 At % of one alloying element from the group comprising calcium, zinc, manganese, lithium, and iron, or up to 50 At % of a combination thereof

5. The implant according to claim 3, wherein the magnesium alloy additionally contains up to 20 At % yttrium or other rare earth elements.

6. The implant according to claim 1, wherein the coating has a layered structure.

7. The implant according to claim 6, wherein the coating has at least one crystalline layer and/or amorphous layer, wherein the crystalline layer and/or the amorphous layer have a layer thickness of 0.1 to 5 microns.

8. The implant according to claim 7, wherein the crystalline layer completely covers the surface of the implant and the amorphous layer covers the crystalline layer.

9. The implant according to claim 7, wherein the amorphous layer has pores with a pore size between 0.02 and 50 microns.

10. The implant according to claim 7, wherein the amorphous layer comprises hydroxy apatite and has pores with a pore size in the range of 0.02 to 1 micron.

11. The implant according to claim 9, wherein the pores of the amorphous layer are tilled with one or more lipids, optionally fatty acids, mono-, di- or triacylglycerides, waxes, phospholipids, sphingolipids, steroids and degradable polymers showing a non-acidic break-down mechanism.

12. The implant according to claim 11. wherein a drug and/or a pH buffer is embedded into the one or more lipids, optionally an anti-proliferative and/or anti-inflammatory drug.

13. The implant according to claim 1. wherein the implant is a stent.
